(19) United States
(12) Patent Application Publication
(10) Pub. No.: US 2024/0219386 A1
(43) Pub. Date: Jul. 4, 2024

(54) DEVICE FOR THE ELECTRONIC AND ELECTROCHEMICAL MEASUREMENT OF ANALYTE CONCENTRATIONS IN BIOLOGICAL SAMPLES

(71) Applicant: PONTIFICIA UNIVERSIDAD JAVERIANA, Bogotá (CO)

(72) Inventors: Andrés JARAMILLO BOTERO, Cali (CO); Drochss VALENCIA, Jamundí (CO); Sammy PERDOMO, Cali (CO); José Hernando MOSQUERA, Cali (CO); Juan David CONTRERAS, Jamundí (CO)

(21) Appl. No.: 18/557,921

(22) PCT Filed: Apr. 27, 2022
(86) PCT No.: PCT/IB2022/053929

§ 371 Date: Oct. 27, 2023

(30) Foreign Application Priority Data
Apr. 27, 2021 (CO) NC2021/0005504

(51) Int. Cl. G01N 33/569; G01N 27/414; G01N 33/543

(52) U.S. Cl. CPC G01N 33/56983; G01N 27/4145; G01N 33/5438; G01N 2333/165; G01N 2469/10

(57) ABSTRACT

The present disclosure refers to an electronic biological sample measurement device that uses at least two detection techniques in virtual concurrence to measure the concentration of a target analyte in at least one biological sample. The detection techniques are electrochemical treatment, electro-optical treatment, and field-effect treatment, or combinations in sequence thereof. The measurement device of the present disclosure may combine at least two selected measurement techniques (and up to three techniques) without manually modifying the connections of said device, which is an advantage in that it improves selectivity and sensitivity in the measurement of the analyte.

### FIELD OF THE DISCLOSURE

The present disclosure relates to devices for measuring analyte concentrations in biological samples. In particular, the present disclosure refers to devices for the electronic, electrochemical, and electro-optical measurement of analyte concentrations in biological samples.

### DESCRIPTION OF THE PRIOR ART

A wide variety of analyte detection and measurement devices are available in the industry for at least one target analyte in at least one biological sample. Analyte detecting devices are configured to reliably detect chemical and/or biological species, qualitatively and/or quantitatively, and can be used for various purposes, including, without limitation, diagnostic purposes, state monitoring and supervision, quality control, or industrial process control; on solid, liquid, or gaseous samples. Such analyte detectors may rely on, e.g., transistor-based measurements to identify at least one analyte. Similarly, there are analyte detectors that use electrochemistry to identify at least one analyte. Transistor-based analyte detectors and electrochemical analyte detectors have been adapted to allow the detection of a wide range of analytes including biomolecules such as proteins, antibodies, antigens, DNA, and chemical species such as ionic species and electrolytes.

However, at present, there is a need in different industries to be more accurate and faster in the detection of analytes. Therefore, different improvements have been made in analyte detection devices, such as the development of devices that use different detection techniques.

Disclosures such as patents US20190376926A1, U.S. Pat. No. 8,778,269B2, U.S. Ser. No. 10/739,304B2 and a non-patent document titled “NO2 sensor with a graphite nanopowder working electrode” are identified in the prior art, which relate to analyte detection devices that perform different detection techniques.

Patent US20190376926A1 discloses an analyte detector for detecting at least one analyte in at least one fluid sample. The analyte detector comprises at least one multipurpose electrode that can be exposed to the fluid sample. The analyte detector further comprises at least one field-effect transistor in electrical contact with the multipurpose electrode. The analyte detector further comprises at least one electrochemical measurement device configured for performing at least one electrochemical measurement using the multipurpose electrode.

In particular, this document discloses the analyte detector for detecting at least one analyte in at least one fluid sample, the analyte detector comprising at least one multipurpose electrode that may be exposed to the fluid sample. Moreover, the analyte detector further comprises at least one field-effect transistor in electrical contact with at least one multipurpose electrode.

On the other hand, the analyte detector of this document further comprises at least one electrochemical measurement device configured to perform at least one electrochemical measurement using the multipurpose electrode.

Moreover, the analyte detector has at least one controller connected to the field-effect transistor and the electrochemical measurement device. Said controller is configured to perform at least one measurement using the field-effect transistor, wherein the controller is further configured to control at least one electrochemical measurement using the electrochemical measurement device.

Specifically, the document mentions that the multipurpose electrode can be at least partially identical to the field-effect transistor channel. On the other hand, it mentions that for glucose sensing using electrochemical measurement, the multipurpose electrode can additionally be employed as a working electrode.

Patent U.S. Pat. No. 8,778,269B2 discloses nanoelectronic devices for the detection and quantification of biomolecules. In certain embodiments, the devices are configured to detect and measure blood glucose levels. Methods of fabricating nanoelectronic devices for the detection of biomolecules are also provided.

In particular, this document discloses the device that comprises one or more conductive elements or contacts that can be placed on the substrate and electrically connected to the conductive channel. The conductive elements may comprise metal electrodes in direct contact with the conducting channel. Alternatively, a conductor or semiconductor material can be interposed between contacts by the conducting channel. The conductive elements may comprise a source electrode S and a drain electrode D upon application of a selected and/or controllable source-drain voltage Vsd (the voltage and/or polarity of the source relative drain can be variable, and said source can be DC, AC and/or pulsed, and the like). In such case, the contacts are arranged so that the nanotube network comprises at least one conductive path between at least one pair of conductors.

Furthermore, this document discloses that a contact or electrode can be employed to provide a charge to the channel relative to a second electrode, such that there is an electrical capacitance between the second electrode and the channel. The second electrode can be a gate electrode, a discrete bottom electrode (e.g., embedded in, under, and/or doped within the substrate), a top gate electrode, a liquid medium electrode, and the like. In another exemplary preferred embodiment, the gate electrode is a conducting element in contact with a conducting liquid, said liquid being in contact with the nanotube network. In other embodiments, the device includes a counter electrode, reference electrode and/or pseudo-reference electrode.

Patent U.S. Ser. No. 10/739,304B2 discloses an organic transistor-based system for electrophysiological monitoring of cells. The system includes a plurality of organic transistors, each comprising: a floating gate electrode; a source electrode and a drain electrode; an organic semiconductor; an insulating layer; and a sensing area. A barrier mechanically separates said sensing area and a transistor area. Each organic transistor includes a control gate electrode coupled to a portion of said floating gate electrode external to said sensing area by a capacitor. The control gate electrode is separated from said floating gate electrode by said insulating layer. The control gate electrode sets a working point of the organic transistor to which the control gate electrode belongs by a control voltage (VGS) applied to it. In each organic transistor, an overlapping area defined by said control gate electrode formed above said floating gate is comprised between 9*10⁻⁴ cm² and 2*10−3 cm².

Moreover, this document discloses that the system may include multichannel (16 channels) dedicated readout and conditioning electronic. Each channel comprises three main blocks: a first inverting I/V converter with a 1 MΩ feedback resistor, a 2^{nd} order high-pass Butterworth filter with a cut-off frequency of 150 Hz, a 3^{rd} order low-pass Butterworth filter with a cut-off frequency of 1.3 kHz. The polarization takes place by means of an adjustable biasing circuit for each of the OCMFETs polarization (VDS=VGS=about −1 V for all the reported measurements, with small variations from transistor to the other). The total voltage gain of the circuit is 110. The realized custom circuit is connected to a multichannel systems acquisition board for A/D conversion, acquisition, and storage. All the measurement sessions were performed inside a Faraday cage in order to minimize electrical environmental noise on the system.

Finally, the non-patent document entitled “NO2 sensor with a graphite nanopowder working electrode” discloses a NO2 measurement device consisting of a graphene field-effect transistor (GFET) covered by the ionic liquid (in the form of solid electrolyte) and graphite nanopowder (GNP) as the working electrode.

In particular, this document discloses that the potential between the working and drain electrodes was set at −250 mV and the drain current was measured to monitor the response of the sensor. The measurement was carried out with a potentiostat connected to the electrodes. The source electrode was used as a pseudo-reference electrode. The back-gate voltage and the drain-source voltage were set to 0 V during measurements of sensing response. The pulse function was used to suppress the heating of the sensor and leads.

However, the prior art does not disclose detection devices that facilitate the use of several measurement techniques with the same device and that improve analyte detection accuracy by merging signals of different types and techniques.

### BRIEF DESCRIPTION OF THE DISCLOSURE

The present disclosure refers to a device for electronic, electrochemical, and optical measurement of analyte concentrations in biological samples, comprising a first electrode and a second electrode, at least one selector circuit connected to the first electrode and second electrode. Moreover, the device comprises an electronic signal-conditioning module connected to at least one selector circuit, an analog-to-digital converter connected to the electronic signal-conditioning module, and a computing unit connected to the analog-to-digital converter and the electronic signal-conditioning module, said computing unit controlling at least one selector circuit.

In particular, the computing unit is configured to transmit electrical signals to the electronic signal-conditioning module and at least one selector circuit is configured to select from the electrodes. Moreover, the first electrode is configured to bind specific analytes of the biological sample.

### BRIEF DESCRIPTION OF THE DRAWINGS

FIG. 1A shows an embodiment of a device for electronic and electrochemical measurement of analyte concentrations in biological samples comprising an electrode configurable as a semiconductor channel of a field-effect transistor or as a working electrode in an electrochemical measurement device, a computing unit for measuring the change in an electrical response signal on the electrode due to the effect of an electronic field induced by the analytes absorbed on its surface, and alternatively configurable for the measurement of electrochemical variables characterizing the organic layer, an electronic signal-conditioning module, a selector circuit and an analog-to-digital converter.

FIG. 1B shows an embodiment of a device for electronic and electrochemical measurement of analyte concentrations in biological samples comprising an electrode configurable as a semiconductor channel of a field-effect transistor or as a working electrode in electrochemical measurement device, a computing unit for measuring the change in an electrical response signal on the electrode due to the effect of an electronic field induced by the analytes absorbed on its surface, and alternatively configurable for the measurement of electrochemical variables characterizing the organic layer, an electronic signal-conditioning module, a selector circuit, a signal generator, an analog-to-digital converter and a Faraday cage.

FIG. 2 shows an embodiment of a device for electronic, electrochemical, and optical measurement of analyte concentrations in biological samples, wherein the device is configured as a field-effect transistor with three electrodes, a first electrode, a second electrode, a four electrode, which acts as a drain, and a contact connected to a portion of the first electrode which acts as a source. The electrode system is printed on a transparent substrate. Moreover, the device comprises an emitter and detector placed under the transparent substrate.

FIG. 3 shows an embodiment of a device for electronic and electrochemical measurement of analyte concentrations in biological samples comprising an electronic signal-conditioning module, which comprises an input-conditioning circuit and an output-conditioning circuit. Moreover, the device comprises a first electrode, a second electrode, a third electrode, a receiver, an input-selector circuit connected to the second electrode and the fourth electrode, and an output-selector circuit connected to the first electrode and the receiver.

FIG. 4 shows a block diagram where a switched reading is made of electrical signals received by an input-selector circuit which is a demultiplexer and an output-selector circuit which is a multiplexer, where the input electrical signals are conditioned by an input-conditioning circuit and the output electrical signals are conditioned by an output-conditioning circuit. The conditioned electrical output signals are delivered to a single analog-to-digital converter. Moreover, a block diagram is shown where a simultaneous reading of the electrical signals received and delivered by the selector circuit is performed, where the input electrical signals are conditioned by an input-conditioning circuit and the output electrical signals are conditioned by an output-conditioning circuit. The conditioned electrical output signals are delivered to analog-to-digital converters.

FIG. 5 shows a graph comparing electrochemistry data obtained from a biological sample with target analytes to electrochemistry data from a biological sample without target analytes.

FIG. 6 shows an embodiment of a device for electronic and electrochemical measurement of analyte concentrations in biological samples at ultra-low concentrations (fg/ml) comprising a Faraday cage and a display device.

### DETAILED DESCRIPTION OF THE DISCLOSURE

The present disclosure is aimed at a device for the electronic and electrochemical measurement of analyte concentrations in biological samples, having a modified electrode for measuring the concentration of biological samples in fluids, which combines different techniques for the selective detection of specific molecules in a biological sample.

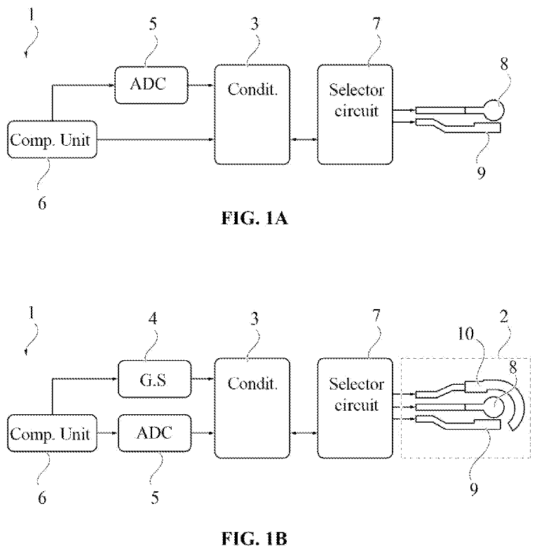

Regarding FIG. 1A, in one embodiment of the disclosure, the device for electronic and electrochemical measurement of analyte concentrations in biological samples (1) comprises:
a first electrode (8) and a second electrode (9);
at least one selector circuit (7) connected to the first electrode (8) and a second electrode (9);
an electronic signal-conditioning module (3) connected to the at least one selector circuit (7);
an analog-to-digital converter (5) connected to the electronic signal-conditioning module (3); and
a computing unit (6) connected to the analog-to-digital converter (5) and to the electronic signal-conditioning module (3), said computing unit (6) controlling at least one selector circuit (7).

In particular, the computing unit (6) can be configured to transmit electrical signals to the electronic signal-conditioning module (3) and the at least one selector circuit (7) is configured to select from the electrodes. Moreover, the first electrode (8) is configured to bind specific analytes of the biological sample.

The device for electronic and electrochemical measurement of analyte concentrations in biological samples (1) combines different techniques that can be selected from electrochemical treatment, electro-optical treatment and field-effect treatment, or combinations in sequence thereof. The above is performed through the selection of electrode configurations according to the technique used. One of the technical effects of combining the electrochemical technique, the field-effect technique and the electro-optical technique is that more precise and complementary results are obtained, compared to the results of each technique separately.

In one embodiment of the disclosure, the computing unit (6) controls the selector circuit (7) so that it selects the first electrode (8) and the second electrode (9), this configuration means that the device for electronic and electrochemical measurement of analyte concentrations in biological samples (1) uses an electrochemical treatment technique on the first electrode (8). There are various electrochemical treatment techniques that can be used by the device of the present disclosure, such as cyclic voltammetry, differential pulse voltammetry, chronoamperometry, impedanciometry, etc. In one example, one of the techniques is electrochemical impedance spectroscopy, which consists of measuring the offset of an oscillatory voltage signal over a range of frequencies in response to the oxidation/reduction process of an electroactive substance, or the charge transfer at the interface of a chemically modified electrode to bind analytes of interest and the electroactive substance.

In one embodiment of the disclosure, the computing unit (6) controls the selector circuit (7) so that it selects the first electrode (8) and disconnects the second electrode (9), which means that the device for electronic and electrochemical measurement of analyte concentrations in biological samples (1) uses an electronic field-effect measurement technique on the first electrode (8). In this embodiment, the first electrode (8) works as a transistor. In particular, a portion of the first electrode (8) receives an electrical signal which can be a direct-voltage signal (e.g., 5 V DC) from the computing unit (6) or indirectly through the analytes and a different portion of the first electrode (8) is connected to ground. In this case, it shall be understood that one electrode, which corresponds to a gate, is immersed in the solution of the analyte to be measured. The analytes that are absorbed on the surface of the first electrode (8), which is a semiconductor element, act as a second gate in the transistor. As mentioned before, the selector circuit (7) switches off the second electrode (9).

Also, the device for electronic and electrochemical measurement of analyte concentrations in biological samples (1) can enable electro-optical measurement techniques such as fluorescence spectroscopy techniques or surface plasmon resonance spectroscopy techniques. Electro-optical techniques may be applied in concurrence with electrochemical or field-effect techniques or can be applied independently.

In one embodiment of the disclosure, the fluorescence spectroscopy technique employs labeling of the analyte with low molecular weight fluorophores. It is important to stress that the presence of a fluorophore allows the use of the ratio of fluorescence intensities to quantitatively detect the level or concentration of the selected analyte molecule present in a solution, as well as its spatial distribution (e.g., agglomeration or dispersion), in conjunction with electrochemical and electrical field-effect techniques, to discriminate the distribution of analytes with minor chemical variations (in one example, structural proteins of different species in a virus).

In another embodiment of the disclosure, no analyte marking is required for surface plasmon resonance spectroscopy, since the analyte rests on the first electrode (8) which can be a channel or electrode with a semi-metallic character (e.g. graphene) and the presence of the analyte causes a change in the refractive index of the material of the first electrode (8) and an offset in the resonance frequency of the surface plasmon waves generated by an electro-optical excitation that is applied.

Optionally, the device for electronic and electrochemical measurement of analyte concentrations in biological samples (1) may have additional electrodes for performing the techniques employed by the device. In one embodiment of the disclosure, the device for electronic and electrochemical measurement of analyte concentrations in biological samples (1) may have three electrodes or four electrodes for performing the measurement techniques.

Regarding FIG. 1B, the device for electronic and electrochemical measurement of analyte concentrations in biological samples (1) further comprises a third electrode (10) which is connected to the at least one selector circuit (7) or to the electronic signal-conditioning module (3).

Preferably, the third electrode (10) can be connected to the signal-conditioning module (3).

The selector circuit (7) can select the first electrode (8), the second electrode (9) and the third electrode (10) automatically, according to a switching signal delivered by the computing unit (6) to the selector circuit (7), according to the detection technique to be used by the device for electronic and electrochemical measurement of analyte concentrations in biological samples (1). Optionally, a first switching signal relates to electrochemical techniques and a second switching signal relates to the field-effect technique.

In one embodiment of the disclosure, in the configuration when the device of the present disclosure has the first electrode (8), the second electrode (9) and the third electrode (10), once the computing unit (6) delivers the first switching signal, wherein said first switching signal indicates that the technique to be used is the electrochemical technique and with regard to FIG. 1B, the electrodes may correspond to: the first electrode (8) corresponds to the working electrode which is the surface on which the reduction/oxidation reactions occur; the third electrode (10) corresponds to the reference electrode, which allows for measuring a reference voltage to the solution containing the analyte to be measured; and the second electrode (9) corresponds to the counter electrode or auxiliary electrode, which serves to close the current circuit in the electrochemical cell, i.e., when the selector circuit (7) receives the first switching signal, it selects the first electrode (8), the second electrode (9) and the third electrode (10).

In turn, in one embodiment of the disclosure, when the computing unit (6) delivers the second switching signal, i.e., when the technique is electronic field-effect measurement, the electrodes can work as a transistor and correspond, respectively, to: the first electrode (8) makes a semiconductor channel, a portion of the electrode (8) is connected to a source contact which fulfills the function of source electrode; similarly, another portion of the first electrode (8) can be connected to ground by means of a drain contact, to work as a drain electrode, said drain electrode establishes a stable potential with respect to the voltage between a gate and the source electrode. On the other hand, the third electrode (10) corresponds to the gate that regulates the transport and electronic switching zone of the semiconductor channel, i.e., when the selector circuit (7) receives the second switching signal, it selects the first electrode (8) and the third electrode (10) and disconnects the second electrode (9).

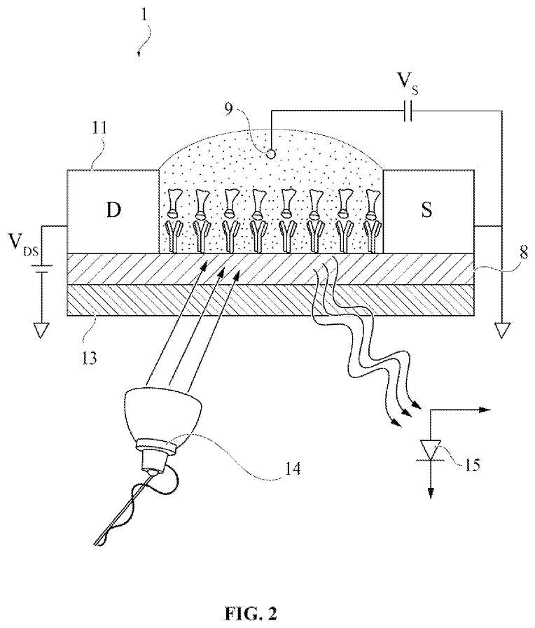

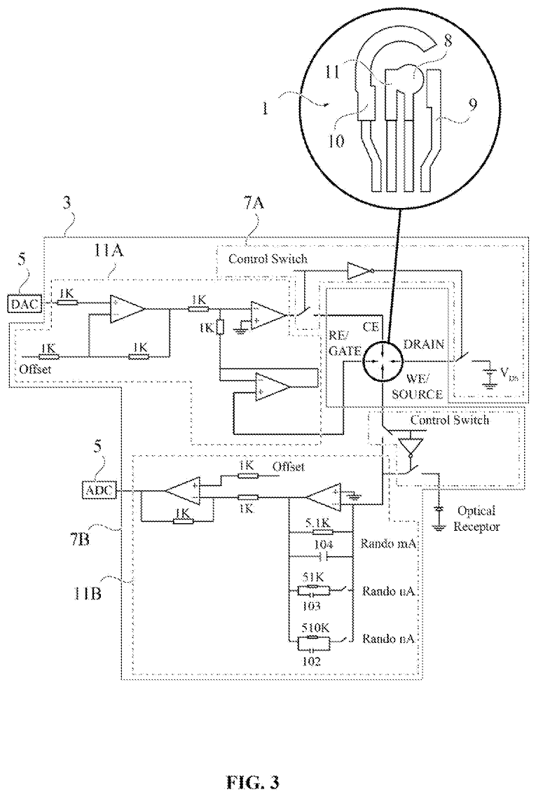

Regarding FIGS. 2 and 3, in one embodiment of the disclosure, the device for electronic and electrochemical measurement of analyte concentrations in biological samples (1) further comprises a fourth electrode (11) connectable to the at least one selector circuit (7), said fourth electrode (11) being connected to the first electrode (8) and to the ground.

The selector circuit (7) can be configured to select the fourth electrode (11) when receiving the second switching signal and disconnect the fourth electrode (11) when receiving the first switching signal, wherein when the fourth electrode (11) is connected, the first electrode (8) is connected to ground and when the fourth electrode (11) is disconnected, the first electrode (8) is disconnected from ground.

Optionally, the fourth electrode (11) connects a portion of the first electrode (8) to ground by means of a capacitor.

In this case, in one embodiment of the disclosure, when the computing unit (6) delivers the first switching signal, i.e., when the electrochemical treatment is to be used, the electrodes may correspond to: the first electrode (8) corresponds to the working electrode which is the surface on which the reduction/oxidation reactions take place; the third electrode (10) corresponds to the reference electrode, which allows for measuring a reference voltage to the solution containing the analyte to be measured; and the second electrode (9) corresponds to the counter electrode or auxiliary electrode, which serves to close the current circuit in the electrochemical cell, i.e., when the device for the electronic and electrochemical measurement of analyte concentrations in biological samples (1) has the first electrode (8), the second electrode (9), the third electrode (10) and the fourth electrode (11), when the selector circuit (7) receives the first switching signal, it selects the first electrode (8), the second electrode (9), the third electrode (10) and disconnects the fourth electrode (11).

On the other hand, in one embodiment of the disclosure, when the computing unit (6) delivers the second switching signal, i.e., when it is a measurement technique by electronic field effect, the electrodes work as a transistor. In this case and regarding FIG. 2, the electrodes may correspond respectively: the first electrode (8) which acts as a semiconductor electrode, connected to a source contact. On the other hand, the third electrode (10) corresponds to a gate that regulates the transport and electronic switching zone of the semiconductor channel. Moreover, in this particular example, the fourth electrode (11), which acts as a drain electrode, is connected to the first electrode (8) and to ground, i.e., when the device for electronic and electrochemical measurement of analyte concentrations in biological samples (1) has the first electrode (8), the second electrode (9), the third electrode (10) and the fourth electrode (11), when the selector circuit (7) receives the second switching signal, it selects the first electrode (8), the third electrode (10), the fourth electrode (11) and disconnects the second electrode (9).

The first electrode (8), the second electrode (9), the third electrode (10) and the fourth electrode (11) can be placed on soft materials or materials synthesized by material, chemical or physical deposition techniques or on hard substrates. Optionally, the first electrode (8), the second electrode (9), the third electrode (10) and the fourth electrode (11) are placed on a substrate (13) which can be silica SiO2.

In one embodiment of the disclosure, the first electrode (8), the second electrode (9) and the third electrode (10), are independent electrodes. Similarly, the fourth electrode (11) can be an independent electrode. In one embodiment of the disclosure, the first electrode (8), the second electrode (9) and the third electrode (10) can be conductive inks. In turn, the fourth electrode (11) can be a conductive ink.

In a particular example, the first electrode (8) is a carbon semiconductor element. In another particular example, the first electrode (8) and the third electrode (10) are printed with conductive carbon or oxide graphene inks on the substrate (13) and the second electrode (9), and the contacts were printed with a conductive Ag/AgCl ink on the substrate (13). In one embodiment of the disclosure, the first electrode (8), the second electrode (9), the third electrode (10) and the fourth electrode (11) can be fabricated in layers. In a particular example, the first electrode (8) is a biomodified electrode that is fabricated with layers of elements, said layers of elements that allow for measuring the interaction between the first electrode (8) and the target analyte in a sample.

In one embodiment of the disclosure, the first electrode (8) is a semiconductor element which can be selected from the group consisting of an activated carbon electrode, doped graphene electrode or graphene in arm-chair type ribbons, or another electrode of a semiconductor element known to a person of ordinary skill in the art.

As mentioned before, in a particular example, when the device for electronic and electrochemical measurement of analyte concentrations in biological samples (1) uses electronic field-effect treatment, the first electrode (8) may correspond to a semiconductor channel. In this particular example, when the first electrode (8) behaves as a semiconductor channel, the distance between the contact of the source electrode and the drain electrode must respect a minimum of 6 nm on the surface of the first electrode (8) or semiconductor channel. The source electrode and the drain electrode can be two contacts, or it can be one contact and the fourth electrode (11) which can act as drain electrode. The minimum distance indicated is intended to ensure thermionic conduction (i.e., to avoid electronic tunneling) through the semiconductor channel, i.e., through the first electrode (8). Both activated carbon and doped graphene or graphene in arm-chair type ribbons have band gap and therefore act as semiconductors. It shall be understood that any material that complies with this characteristic can be used as material for the first electrode (8).

In order to bind specific analytes of the biological sample, the first electrode (8) can be a biomodified electrode, wherein in a particular example, the first electrode (8) is a conductive carbon ink on a polymer substrate, a layer of chemically related ligands to bind on one end to the substrate and on the other to a protein via a peptide bond (in one implementation, the diazonium salt mixture of 4-aminobenzoic acid), an antibody that binds to the ligand surface via a peptide bond, and in a non-covalent manner to a protein of the target analyte (e.g., a structural protein of SARS COV-2).

In one embodiment of the disclosure, the device for electronic and electrochemical measurement of analyte concentrations in biological samples (1) comprises a shielding enveloping the first electrode (8) and the second electrode (9), which allows the removal of electromagnetic interference on the measurement signals. In another embodiment of the disclosure, the shielding surrounds the first electrode (8), the second electrode (9) and the third electrode (10). In a particular example, the shielding involves the first electrode (8), the second electrode (9), the third electrode (10) and the fourth electrode (11). In another particular example, the shielding is a Faraday cage.

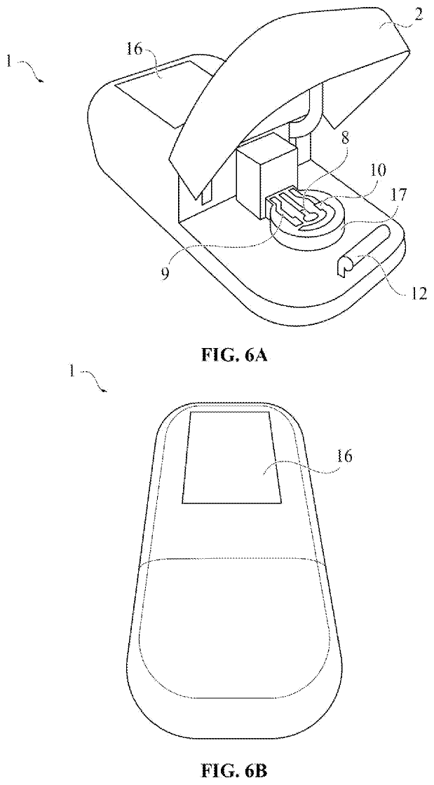

Regarding FIGS. 1B and 6, the device for electronic and electrochemical measurement of analyte concentrations in biological samples (1) further comprises a Faraday cage (2) enclosing the first electrode (8), the second electrode (9) and the third electrode (10), which allows for minimizing electrical environmental noise in the device. Regarding FIG. 6, it is observed that the Faraday cage (2) is sealed by means of a flange (12).

On the other hand, the electronic signal-conditioning module (3) can be configured to send conditioned electrical signals depending on the technique to be used, e.g., in the case where electrochemical treatment is to be used, the electronic signal-conditioning module (3) sends a first electrical signal to the first electrode (8). In one embodiment of the disclosure, the first electrical signal is a sinusoidal voltage signal going between the first electrode (8), which corresponds to the working electrode and the third electrode (10), which corresponds to the reference electrode, said sinusoidal voltage signal may have a 10 mV amplitude in a frequency range between 0.02 Hz and 20 KHz. In this case, the first electrode (8) generates an electrical signal response of the electrochemical treatment, said electrical signal of response of the electrochemical treatment is an alternating voltage and current signal between the first electrode (8) and the third electrode (10), which may have a sinusoidal form at the same frequency as the first electrical signal, but shifted in phase. The electronic current flowing from the third electrode (10) (which in this example is the reference electrode) to the first electrode (8) (which in this example is the working electrode), as a result of the application of the sinusoidal voltage signal in the given frequency range, allows the calculation of the equivalent impedance of the first electrode (8) in the electrochemical cell.

On the other hand, and as mentioned before, when the device for electronic and electrochemical measurement of analyte concentrations in biological samples (1) is going to use the electronic field-effect treatment, the selector circuit (7) applies a second electrical signal in a portion of the first electrode (8) which can be a DC voltage signal and it is grounded in another different portion of the first electrode (8). In a particular example, the grounding is made by means of the fourth electrode (11) corresponding to the drain electrode. In turn, the third electrode (10) is used to modulate the electronic conductivity of the first electrode (8) in its linear zone.

The absorption of the target analyte on the selectivity-modified surface of the first electrode (8) alters the transmission spectrum of the semiconductor channel which is the first electrode (8), i.e., doping it positively or negatively depending on the target analytes. This doping produces an offset in the minimum of the transconductance curve of the device for the electronic and electrochemical measurement of analyte concentrations in biological samples (1). The above allows the confirmation of minimal changes in the protein concentration of the target analytes. Indeed, in the field-effect treatment the first electrode (8) generates an electrical response signal, and this electrical response signal can be a current signal and can be digitally complemented with the electrical response signal of the electrochemical treatment, which may also be a current signal, and without the need to change the device or connections.

In a particular example and as mentioned before, when using the electronic field-effect treatment, the second electrode (9) can be disconnected. In another particular example, when the electronic field-effect treatment is used, the third electrode (10), which is immersed in electrolytic solvent, operates to amplify the electron flow between a portion of the first electrode (8) (source) and another portion of the first electrode (8) (drain) which is grounded.

Optionally, in the electro-optical technique, the solution with the target analyte is conjugated with a chemically related fluorophore to determine the spatial distribution and concentration of the analyte, washed with pure water to remove excess fluorophore molecules in the solution and excited by fast-pulsed radiation (femtoseconds) in the range between lower frequency ultraviolet and infrared. For the above, the device for electronic and electrochemical measurement of analyte concentrations in biological samples (1) has an emitter (14) and a detector (15), in this case the device can have the first electrode (8) and the second electrode (9) to perform the electrochemical, field-effect and electro-optical technique.

Optionally, the device for electronic and electrochemical measurement of analyte concentrations in biological samples (1) has the emitter (14) and the detector (15), in this case the device can have the first electrode (8), the second (9) and the third electrode (10) to perform the electrochemical, field-effect and electro-optical technique.

Regarding FIG. 2, in one embodiment of the disclosure, when the device for electronic and electrochemical measurement of analyte concentrations in biological samples (1) is going to use the electro-optical technique, the device for electronic and electrochemical measurement of analyte concentrations in biological samples (1) may further comprise the emitter (14) configured to radiate at the excitation wavelength of the fluorophore employed for labeling. Moreover, the device may further comprise the detector (15) configured to sense the wavelength of fluorescence emitted by the labeling fluorophore or to count photons or sense the resonance frequency of the surface plasmon according to electro-optical technique. In this embodiment of the disclosure, the substrate (13) and the first electrode (8) should be optically transparent, e.g., the substrate (13) can be silica and the first electrode (8) can be graphene doped or cut into semiconductor ribbons. As observed in FIG. 2, in one embodiment of the disclosure, the emitter (14) and detector (15) are located below the substrate (13).

In a non-illustrated embodiment, the emitter (14) and detector (15) are placed above the substrate (13).

The emitter (14) can be configured to emit radiation at a particular wavelength, said wavelength can be ultraviolet, infrared, or visible. Optionally, the emitter (14) can be configured to emit radiation at an infrared or visible wavelength. Alternatively, the emitter (14) can be configured to emit radiation at an ultraviolet wavelength, in this case by fast-pulsed radiation (between 10 and 500 femtoseconds) at a low wavelength. Optionally, the emitter (14) is configured to emit radiation at a wavelength between 350 nm to 1000 nm, even more preferably, between 450 nm to 550 nm. In a particular example, the emitter (14) is an LED. Optionally, the emitter (14) can be connected to the computing unit (6), said computing unit (6) controlling the wavelength radiated by the emitter (14).

In turn, the detector (15) can be configured to receive a response radiation from the interaction of the radiation emitted by the emitter (14) with the first electrode (8) and to generate an electrical response signal and to deliver it to the electronic signal-conditioning module (3). Optionally, the detector (15) can be configured to detect a radiation at a wavelength between 350 nm to 1000 nm, even more preferably, between 450 nm to 550 nm.

The detector (15) can be selected from the group consisting of a photon counter (at single or multiple frequencies), an angle meter for determining the change in resonant frequency of a surface plasmon, a luminous intensity sensor or other elements used to perform electro-optical techniques known to a person of ordinary skill in the subject-matter, and combinations of the foregoing. In a particular example, the detector (15) is a luminous intensity sensor. In another particular example, when analyte tagging is not used, the detector (15) is an angle meter for determining the change in resonance frequency of a surface plasmon.

In one embodiment of the disclosure, the detector (15) can be connected to the at least one selector circuit (7), wherein the at least one selector circuit (7) selects the detector (15) and disconnects the first electrode (8) and the second electrode (9) or may select the detector (15), the first electrode (8) and disconnect the second electrode (9) or may select the detector (15), the first electrode (8) and the second electrode (9). One of the technical effects of the above is to allow a separate technique which can be electrochemical, field-effect or electro-optical technique to be performed with a different technique. On the other hand, the detector (15) being connected to a selector circuit (7) allows the electro-optical technique to be performed at the same time with another technique that can be the electrochemical or the field-effect technique. Finally, the selector circuit (7) may allow the three techniques to be performed independently in a particular order. In another embodiment of the disclosure, the emitter (14) and the detector (15) can be connected to the at least one selector circuit (7), wherein the at least one selector circuit (7) selects the detector (15), the emitter (14) and disconnects the first electrode (8) and the second electrode (9) or may select the detector (15), the emitter (14), the first electrode (8) and disconnect the second electrode (9) or may select the detector (15), the first electrode (8) and the second electrode (9).

It shall be understood that in embodiments where the emitter (14) and/or the detector (15) are connected to a selector circuit (7), said selector circuit (7) may also be connected to the third electrode (10) and/or the fourth electrode (11).

The electronic signal-conditioning module (3) may manage the type of test and the corresponding circuit to act on the electrodes, filters and pre-amplifies and/or amplifies the signals obtained between the electrodes and processes them depending on the selected technique. In particular, in the device for electronic and electrochemical measurement of analyte concentrations in biological samples (1) the signal to be amplified is in the order of nanoamperes in current and millivolts in potential.

It shall be understood for the present disclosure by signal conditioning, the actions selected from the group formed to separate, combine, or transform signals of current, voltage, frequency, impedance, current and combinations thereof.

The electronic signal-conditioning module (3) may comprise at least one conditioning circuit, wherein the conditioning circuits are responsible for conditioning the electrical input signals and the electrical response signals of the first electrode (8) and/or the detector (15).

In turn, the at least one selector circuit (7) is responsible for selecting the different electrodes or other elements connected to it (e.g., detector (15), emitter (14)) depending on the switching signal received by the computing unit (6).

The selector circuit (7) can be a circuit made up of combinations of the following electronic elements: an analog demultiplexer, an analog multiplexer, transistors (e.g. BJT transistors, MOSFETS transistors, IGBT transistors) configured in cut-off and saturation, relays, logic gates, thyristors, Silicon-Controlled Rectifiers (SCRs), TRIAC diodes, DIAC diodes, optocouplers, analog switches, operational amplifiers or combinations thereof, whose combinations allow the signal coming from the electronic signal-conditioning module (3) to be delivered to or received by the electrodes.

It shall be understood that a demultiplexer is a combinational circuit having an electrical signal input, at least two electrical signal outputs and at least one control input. The control input of the demultiplexer is used to select one of the at least two electrical signal outputs, through which the electrical signal that enters through the electrical signal input is to be output. In turn, it shall be understood that a multiplexer is a combinational circuit having at least two electrical signal inputs, one electrical signal output, and at least one control input serving to select one of the at least two electrical signal inputs, through which an electrical signal is to enter, which is delivered to the electrical signal output.

In a particular example, the selector circuit (7) being an analog demultiplexer or an analog multiplexer, allows the analog electrical signal to exit through the electrical signal output of the demultiplexer or a multiplexer to not incorporate electrical alterations regarding the analog electrical signal entering through one of the electrical signal inputs of the demultiplexer or a multiplexer.

In other words, the use of an analog demultiplexer allows that in the selector circuit (7), the analog signals of its output to be faithful to the analog signals of its inputs. In turn, the use of an analog multiplexer allows the selector circuit (7), the analog signals of its outputs to be faithful to the analog signal of its inputs.

In contrast and to give an illustrating example, if a circuit employing a DIAC diode is used in the selector circuit (7), said circuit may incorporate a voltage drop in the output of a signal entering the selector circuit (7) which does not incorporate the use of an analog demultiplexer.

Regarding FIG. 1B, in one embodiment of the disclosure, the selector circuit (7) can be configured to select the first electrode (8), the second electrode (9) and the third electrode (10) depending on the received switching signal.

Specifically, the at least one selector circuit (7) allows the selection of the first electrode (8), the second electrode (9) and the third electrode (10), whereby it is possible to switch from a first configuration to a second configuration, wherein the first configuration is associated with the electrochemistry technique and the second configuration is associated with the field-effect technique.

In an example of the disclosure, when the computing unit (6) sends the first switching signal to the at least one selector circuit (7) this is placed in the first configuration, i.e., the first electrode (8), the second electrode (9) and the third electrode (10) are selected and the electronic signal-conditioning module (3) applies the first voltage signal to the first electrode (8). When the computing unit (6) delivers the second switching signal to the at least one selector circuit (7) this is placed in the second configuration, i.e., the first electrode (8) and the third electrode (10) are selected and the second electrode (9) is disconnected, in this case, the electronic signal-conditioning module (3) applies a second DC voltage signal to the first electrode (8). Whereby, a portion of the first electrode (8) would go on to operate as the source electrode. Also, a different portion of the first electrode (8) is connected to ground, so that said portion operates as the drain electrode.

Optionally and when the device for electronic and electrochemical measurement of analyte concentrations in biological samples (1) has a fourth electrode (11), a portion of the first electrode (8) is connected to the fourth electrode (11), which would come to act as a drain electrode. In this case, the fourth electrode (11) can be connected to at least one selector circuit (7), wherein when the computing unit (6) delivers the second switching signal the at least one selector circuit (7) selects the first electrode (8), the third electrode (10), the fourth electrode (11) and disconnects the second electrode (9). In turn, when the computing unit (6) delivers the first switching signal the selector circuit (7) selects the first electrode (8), the third electrode (10), the second electrode (9) and disconnects the fourth electrode (11).

Regarding FIG. 3, in one embodiment of the disclosure, the electronic signal-conditioning module (3) comprises an input-conditioning circuit (3A) connected to an input-selector circuit (7A). In particular, the input-selector circuit (7A) is connected to the second electrode (9). Moreover, the electronic signal-conditioning module (3) comprises an output-conditioning circuit (3B) connected to an output-selector circuit (7A). The output-selector circuit (7B) is connected to the first electrode (8).

The input-conditioning circuit (3A) is responsible for conditioning the voltage signals that are delivered by the computing unit (6) according to the measurement technique to be applied. Optionally, the input-conditioning circuit (3A) may comprise at least one operational amplifier, which is configured according to the need of the applied techniques.

In a particular example and regarding FIG. 3, the input-conditioning circuit (3A) may comprise a first operational amplifier connected to the analog-to-digital converter (5), a second operational amplifier connected to the first operational amplifier, and a third operational amplifier connected to the first operational amplifier and the third electrode (10). Regarding FIG. 3, in this particular example, the first operational amplifier is configured as a compensated non-inverting differential amplifier and the second operational amplifier is configured as a voltage comparator and the third operational amplifier is configured as a voltage follower.

Similarly, the electronic signal-conditioning module (3) can be connected to at least one input-selector circuit (7A), said input-selector circuit (7A) being connected to the second electrode (9). The input-selector circuit (7A) is responsible for switching said second electrode (9) on or off according to the switching signal delivered by the computing unit (6). Optionally, the third electrode (10) can be connected to an input-selector circuit (7A). On the other hand, in case the device for electronic and electrochemical measurement of analyte concentrations in biological samples (1) is equipped with the fourth electrode (11), it is also connected to an input-selector circuit (7A).

Optionally, the second electrode (9) and the fourth electrode (11) are connected to a selector circuit (7A) which can be an analog demultiplexer. In this case the second electrode (9) is connected to one of the electrical signal outputs of the analog demultiplexer and the fourth electrode (11) is connected to another of the electrical signal outputs of the analog demultiplexer. In turn, the computing unit (6) is connected to the control input of the analog demultiplexer, and the electrical signal input is connected to the input-conditioning circuit (3A). In this case, the computing unit (6) delivers the first switching signal or the second switching signal to the control input of the analog demultiplexer so that the analog demultiplexer selects one of its electrical signal outputs.

In a particular example and regarding FIG. 3, the electronic signal-conditioning module (3) can be connected to an input circuit (7A) connected to the second operational amplifier, to the second electrode (9) and to the fourth electrode (11), wherein the input circuit (7A) has a voltage inverter. In this particular example, the voltage inverter ensures that with a single switching signal sent by the computing unit (6), the second electrode (9) and the fourth electrode (11) are never selected at the same time.

In turn, the output-conditioning circuit (3B) can be responsible for conditioning the response signals of the first electrode (8). Optionally, the output-conditioning circuit (3B) may comprise at least one operational amplifier.

In a particular example and regarding FIG. 3, the output-conditioning circuit (3B) may comprise a first amplifier that is operationally connected to the first electrode (8) and a second amplifier that is operationally connected to the first operational amplifier and the analog-to-digital converter (5), wherein the first operational amplifier is configured as a gain-controlled integrator and the second operational amplifier is configured as a non-inverting compensated differential amplifier.

Similarly, the electronic signal-conditioning module (3) may have at least one output-selector circuit (7B), said output-selector circuit (7B) is connected to the first electrode (8), the output-selector circuit (7B) is responsible for selecting or disconnecting the first electrode (8).

In a particular example and regarding FIG. 3, the electronic signal-conditioning module (3) can be connected from an output-selector circuit (7B) to the first operational amplifier, to the first electrode (8) and to a detector (15), wherein the output-selector circuit (7B) has a voltage inverter. In this particular example, the voltage inverter ensures that with a single switching signal delivered by the computing unit (6), the first electrode (8) and the detector (15) are never switched on at the same time.

Optionally, the first electrode (8) and the detector (15) are connected to an output-selector circuit (7B) which can be an analog multiplexer. In this case the first electrode (8) is connected to one of the electrical signal inputs of the analog multiplexer and the detector (15) is connected to another of the electrical signal inputs of the analog multiplexer. In turn, the computing unit (6) is connected to the control input of the analog multiplexer and the electrical signal output is connected to the output-conditioning circuit (3B). In this case, the computing unit (6) delivers the first switching signal and/or the second switching signal to the control input of the analog multiplexer so that the analog multiplexer selects one of its electrical signal inputs.

The computing unit (6) is configured to deliver electrical signals to the electronic signal-conditioning module (3), such signals can be in different wave forms such as sinusoidal, square, triangular, ramp, and pulses in a voltage-controlled oscillator. In one embodiment of the disclosure and regarding FIG. 1B, the computing unit (6) has a signal generator (4), which is connected to the electronic signal-conditioning module (3), said signal generator (4) is an electronic device that generates periodic or non-periodic signal patterns both analog and digital.

The signal generator (4) can be analog and digital (e.g., XR2206). In a particular example, the signal generator (4) is an integrated circuit XR2206, which is a configurable signal generator, whose operating principle is based on a voltage-controlled oscillator. The XR2206 integrated circuit is capable of generating square, sinusoidal, and triangular functions.

On the other hand, the analog-to-digital converter (5) is connected to the signal generator (4) and to the electronic signal-conditioning module (3), wherein the analog-to-digital converter (5) is an electronic device capable of converting an analog signal, either voltage or current, into a digital signal by means of a quantizer and coded in many cases in a particular binary code, where a code is the univocal representation of the elements, in this case, each binary numerical value corresponds to a single voltage or current value.

The device for the electronic and electrochemical measurement of analyte concentrations in biological samples (1) has a computing unit (6) which is configured to be connected to the analog-to-digital converter (5) and the electronic signal-conditioning module (3). The computing unit (6) is a device that allows the processing of data coming from an external element such as, e.g., the analog-to-digital converter (5). The computing unit (6) carries out different actions, such as the generation of a switching signal, generation of periodic or non-periodic signal patterns both analog and digital, generation of data, access to a stored data, among other possible actions that can be performed.

The computing unit (6) can be selected from the group consisting of: microcontrollers (e.g., PSOC 4BLE), microprocessors, DSCs (Digital Signal Controllers), FPGAs (Field Programmable Gate Arrays), CPLDs (Complex Programmable Logic Devices), ASICs (Application Specific Integrated Circuits), SoCs (System on Chip), PSoCs (Programmable System on Chip), Raspberry Pi, Raspberry Pi, and other computers, PSoCs (Programmable System on Chip), Raspberry Pi, computers, servers, tablets, cell phones, smart phones, signal generators and computing units, processing units or processing modules known to a person of ordinary skill in the art and combinations thereof. In a particular example, the computing unit is a microcontroller.

Regarding FIG. 6, the computing unit (6) is connected to a display device (16), this corresponds to any device that can be connected to a computing unit and display its output. It is selected among others from CRT (Cathode Ray Tube) monitor, flat panel display, LCD (Liquid Crystal Display) display, active matrix LCD display, passive matrix LCD display, LED displays, display projectors, TV (4KTV, HDTV, plasma TV, Smart TV), OLED (Organic Light Emitting Diode) displays, AMOLED (Active Matrix Organic Light Emitting Diode) displays, QD (Quantum Dot Displays), segment displays, among other devices capable of displaying data to a user, known to technical experts, and combinations thereof.

In addition, the computing unit (6) is connected and/or a Human Interface Device (HID) includes, without limitation, keyboard, mouse, trackball, touchpad, pointing device, joystick, touch screen, among other devices capable of allowing a user to input data into the computing unit of the device and combinations thereof.

The computing unit (6) is wirelessly connected to an external device, wherein the computing unit is configured to deliver analyte detection data to the external device and/or receive operating data from the device of the present disclosure.

In particular, the computing unit (6) is connected to the external device or devices by means of a communications module, which is a hardware element that couples a computing unit, processing unit, or processing module or a server, which is configured to establish communication by means of communication links between one or more computing units or servers to exchange data, commands and/or tags. The communication module is selected from the group consisting of wired communication modules, wireless communication modules, and wired and wireless communication modules.

The wireless communication module uses a wireless communication technology that is selected from the group consisting of Bluetooth, WiFi, RF ID (Radio Frequency Identification), UWB (Ultra-Wide Band), CALM (Communications Access for Land Mobile) standard, GPRS, Konnex or KNX, DMX (Digital MultipleX), WiMax and equivalent wireless communication technologies known to a person of ordinary skill in the art and combinations thereof.

The wired communications module has a wired connection port that allows communication with external devices via a communications bus, which is selected from a group consisting of I2C (IIC Inter-Integrated Circuit), CAN (Controller Area Network), BUS RS-232, BUS RS-485, BUS-422, BUS-423, Ethernet, SPI (Serial Peripheral Interface), SCI (Serial Communication Interface), QSPI (Quad Serial Peripheral Interface), 1-Wire, D2B (Domestic Digital Bus), Profibus and others known to a person of ordinary skill in the art.

The computing unit (6) can be connected to a temperature sensor that is configured to measure the temperature at which each applied technique is performed. One of the technical effects of knowing the temperature during the application of each technique, is that it allows to rule out measurements performed under temperatures that may affect the concentration measurement of analytes in the sample. Optionally, the computing unit (6) can be connected to a cooling system that allows for controlling the temperature at which each technique is performed, the cooling system can be selected from the group consisting of a Peltier cell, fans, heat exchangers, or other systems that allow for cooling down a space known by the person of ordinary skill in the art and combinations thereof.

The device for electronic and electrochemical measurement of analyte concentrations in biological samples (1) may have a case that houses the electrical components (e.g., the computing unit, selector circuit, electronic signal-conditioning module, among others). Regarding FIG. 6, in one embodiment of the disclosure, the case is connected to the Faraday cage (2) by means of a pivotable element and secured to the case by means of the flange (12). On the other hand, the Faraday cage (2) forms a chamber, in said chamber an electrode holder (17) is placed where the substrate (13) with the electrodes is located, said electrode holder (17) is coupled to the case. Moreover, the display device (16) is placed on an outer surface of the case for displaying, in a particular example, a final measurement data obtained by the device for electronic and electrochemical measurement of analyte concentrations in biological samples (1).

The electrode holder (17) may have a coupling element that allows the electrode holder to be removable and thus disposable and may also include channels for handling and storing fluids required for measurements. The coupling element can be tabs.

With the device and embodiments of said device described before, the present disclosure implements methods for detecting the presence of analytes of interest in a target sample.

In general, the method of the present disclosure is based on combining at least two treatment techniques for detecting analytes of interest with the device for electronic and electrochemical measurement of analyte concentrations in biological samples (1), wherein the detection is done without modifying connections manually in said device.

The method for electronic and electrochemical measurement of analyte concentrations in biological samples comprises the following steps:
a) receiving a first switching signal in at least one selector circuit (7), said selector circuit (7) selects a first electrode (8) and a second electrode (9), said first switching signal is generated by a computing unit (6) connected to the selector circuit (7);
b) applying first electrical signals to the first electrode (8) and to the second electrode (9), said first electrical signals are transmitted by an electronic signal-conditioning module (3) which is connected to the first electrode (8), to the second electrode (9) and to the computing unit (6);
c) generating an electrical signal of electrochemical response at the first electrode (8) from the first electrical signals in the presence of a given concentration of analytes;
d) receiving a second switching signal in at least one selector circuit (7), said selector circuit (7) selects the first electrode (8) and disconnects the second electrode (9), said second switching signal is generated by the computing unit (6) connected to the selector circuit (7);
e) applying second electrical signals to the first electrode (8), said second electrical signals are generated by the computing unit (6);
f) generating a field-effect response electrical signal at the first electrode (8) in the presence of a given concentration of analytes;
g) generating final measurement data from the response signals generated in steps c) and f) by means of an analog-to-digital converter (5) connected to the computing unit (6).

Optionally, the first electrical signals may correspond to a sinusoidal voltage signal and the second electrical signals can be a direct current voltage signal.

The first electrical signal is related to the electrochemical technique and the second electrical signal is related to the field-effect treatment. The measurement of the field-effect response electrical signal is obtained directly from the effect of the analytes by the field-effect treatment technique.

In one embodiment of the disclosure, step (g) may have the following sub-steps:
g1) generating electrochemical measurement data from the electrochemical response electrical signal obtained in step c) by means of the analog-to-digital converter (5);
g2) generating a field-effect measurement data from the field-effect response electrical signal obtained in step f) by means of the analog-to-digital converter (5); and
g3) generating final measurement data from the data generated in steps g1) and g2).

In particular, electrochemical measurement data and field-effect measurement data can be obtained by means of a switched reading of the electrical response signals obtained in both techniques. In this case, the device for electronic and electrochemical measurement of analyte concentrations in biological samples (1) has an input-selector circuit (7A) which is a demultiplexer that switches the first electrical signal and the second electrical signal that are delivered to the first electrode (8) according to the reception of the first and second switching signals. Moreover, the device for electronic and electrochemical measurement of analyte concentrations in biological samples (1) has an output-selector circuit (7B) which is a multiplexer that switches the electrochemical response electrical signal and the field-effect response electrical signal so that these electrical signals are sent simultaneously to a single analog-to-digital converter (5).

On the other hand, in one embodiment of the disclosure, the electrochemical measurement data and the field-effect measurement data are obtained by simultaneous reading of the electrical signals obtained in both techniques, wherein the electrical response signals of each technique are delivered to two analog-to-digital converters (5), wherein each analog-to-digital converter (5) is associated with a technique being applied.

Moreover, in the method of the present disclosure some electro-optical measurement data can be generated from the emission of a radiation with a wavelength determined by means of an emitter (14) and the detection of wavelength intensity changes of the sample by means of a detector (15). In this case, the following additional steps are performed:
activating an emitter (14) to emit radiation with a given wavelength onto the surface of the first electrode (8);
detecting intensity changes in the expected frequency of fluorescence by means of a detector (15); and
generating an electrical signal of electro-optical response from the detected intensity changes;

In this particular example, the final measurement data are generated from the electrochemical response electrical signal, the field-effect response electrical signal; and the electrical signal of electro-optical response. In this case, step g) has an additional sub-step to g1) and g2), which corresponds to a step g3) to generate electro-optical measurement data from the electrical signal of electro-optical response obtained by means of the analog-to-digital converter (5).

The steps for generating the electrical signal response electro-optical response can be done simultaneously during steps a) to c), i.e., the generation of electrical signals as electro-optical response is obtained during the steps where the electrical signal electro-optical response is generated. Similarly, the steps for generating the electrical signal of electro-optical response can be performed simultaneously during steps d) to f), i.e., the generation of the electrical signal of electro-optical response is obtained during the steps where the field-effect response electrical signal is generated. Moreover, the response signals of the three techniques can be generated in sequence, i.e., first the electrochemistry response electrical signal is generated, second the field-effect response electrical signal is generated, and finally the electrical signal of electro-optical response is generated.

Optionally and as mentioned before, the detector (15) can be connected to the at least one selector circuit (7), in this case, when the control circuit (7) receives a third switching signal, said selector circuit (7) selects the detector (15) and disconnects the first electrode (8) and the second electrode (9). The third switching signal can be associated with the electro-optical technique.

On the other hand, and in another embodiment of the disclosure, the detector (15) can be connected to the at least one selector circuit (7), in this case, when the control circuit (7) receives a third switching signal, said selector circuit (7) selects the detector (15) and disconnects the first electrode (8), the second electrode (9), the third electrode (10) and the fourth electrode (11).

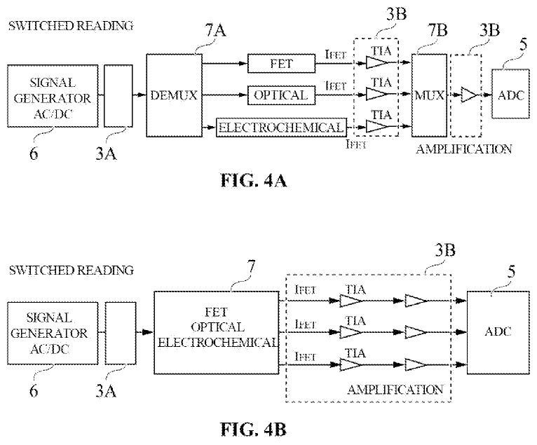

In particular, the switching of the electrical response signals can be performed when performing the three techniques: electrochemical, field-effect and electro-optical, in which case the three response signals obtained in each technique are time-switched and delivered to a single analog-to-digital converter (5). Regarding FIG. 4, in one embodiment of the disclosure, the device for electronic and electrochemical measurement of analyte concentrations in biological samples (1) has an input-selector circuit (7A) which is a demultiplexer that switches the first voltage signal and the second voltage signal that are delivered to the first electrode (8) according to the reception of the first and second switching signals. Moreover, the device for electronic and electrochemical measurement of analyte concentrations in biological samples (1) has an output-selector circuit (7B) which is a multiplexer that switches the electrochemical response electrical signal, the field-effect response electrical signal and the electrical signal of electro-optical response so that these electrical signals are simultaneously sent to a single analog-to-digital converter (5).

Similarly, simultaneous signal reading is possible when performing the three techniques the electrochemical, the field-effect and the electrofluorescence techniques, in this case the three signals obtained are delivered to three analog-to-digital converters (5) or to a single multichannel analog-to-digital converter (5). Regarding FIG. 4, in one embodiment of the disclosure, the electronic signal-conditioning module (3) has a selector circuit (7) that selects the electrodes and/or the detector (15) according to the switching signal that is delivered from the computing unit (6), and the electrical response signals that are generated at the first electrode (8) and at the detector (15) are sent to different analog-to-digital converters (5).

The final measurement data can be obtained by performing an operation between the data obtained from the different measurement techniques, e.g., the operation can be a weighted average of the data obtained from the different measurement techniques in the method of the present disclosure. The final measurement data can be generated by performing the operation between data obtained from two measurement techniques or from three measurement techniques.

In a particular example, the operation to generate the final measurement data is performed on the electrochemical measurement data, the field-effect measurement data and the electro-optical measurement data, in this case, the operation is a weighted average with equal relative weights for the submitted different data obtained by the different measurement technique, i.e. (w1*value of the field-effect measurement data+w2*value of the electrochemical measurement data+w3*value of the electro-optical measurement data)/(w1+w2+w3) for w1=w2=w3. Indeed, for the field-effect measurement, the field-effect measurement data corresponds to a change value, either in current of the characteristic curve, or in the positive or negative doping offset in the transconductance curve. For electrochemical measurements, depending on the type of technique used, the electrochemical measurement data may correspond to a differential value of Rct (charge transfer resistance) in an impedance spectroscopy measurement, or the value of the difference between the current before and after a pulse, in a DPV (differential pulse voltammetry) technique. Finally, in electro-optical measurement, the electro-optical measurement data can correspond to the differential intensity value, before and after the insertion of the analyte. In this particular case, each type of measurement must be normalized over the range of values expected for the particular analyte. One of the technical effects of merging the response signals obtained by three different techniques used is that they allow improved sensitivity (LOD) and selectivity in analyte quantification.

In a particular example, the relative weight of each technique can be varied, for example if in preliminary analyte testing, it is found that the analyte to be measured is more sensitive to the field-effect technique, the relative weight can be increased with respect to the other electrochemical and electro-optical techniques, and that adjustment can be made by supervised case training (i.e. it does not have to be manual).

In one embodiment of the disclosure, the generation of electrochemical measurement data in the computing unit (6) occurs from at least one supervised or unsupervised machine learning algorithm, wherein the at least one machine learning algorithm determines the electrochemical equivalent circuit according to discrete points representing the Nyquist plot calculated by electrochemical impedance spectroscopy. Once the equivalent circuit is determined, the computing unit (6) generates equivalent circuit parameter data corresponding to the equivalent circuit parameters that were determined. Finally, the equivalent circuit parameter data is processed through another learning algorithm, which identifies the parameters and associates with them a concentration of the target analyte, which is represented in the electrochemical measurement data.

Optionally, with the final measurement data a validation can be made to check for the presence of target analytes, such validation can be made, in one embodiment of the disclosure, with the comparison of the generated final measurement data with a predetermined measurement data. In a particular example, the predetermined measurement data represents final measurement data from a sample without target analytes.

Similarly, the method for electronic and electrochemical measurement of analyte concentrations in biological samples may have an additional message generation step. In this message generation step, in one embodiment of the disclosure, the final measurement data can be delivered to the display device (16), which receives the validation data and displays a message to the user (e.g., a text message, a graph).

On the other hand, in this message generation step, in another embodiment of the disclosure, the final measurement data can be compared with a predetermined measurement data and from the comparison the computing unit (6) can generate a validation data. In one embodiment of the disclosure, the validation data is delivered to the display device (16), which receives the validation data and displays a message to the user (e.g., a text message, a graph).

In a particular example and as mentioned before, the computing unit (6) for the generation of electrochemical measurement data may perform the calculation of the impedance of the first electrode (8), said calculation being obtained from the electrochemical response signal generated by the first electrode (8), due to the application of a sinusoidal voltage potential in a given frequency range between the first electrode (8) and the third electrode (10). In this particular example, the computing unit (6) can employ Ohm's law to calculate the impedance at each frequency step, which is defined as:

$$Z = \frac{V}{I} {e}^{i Γ} \qquad ( 1 )$$

Specifically, Γ represents the offset between voltage and current signals. Considering Euler's identity, the real and imaginary components are calculated as follows:

$$Re ( Z ) = \frac{V}{I} cos Γ \qquad ( 2 )$$

$$Im ( Z ) = \frac{V}{I} sen Γ \qquad ( 3 )$$

Where each pair of impedance components (real and imaginary) defined for a specific frequency represents a point in the impedance spectrum that is plotted by a Nyquist or Bode diagram representation.

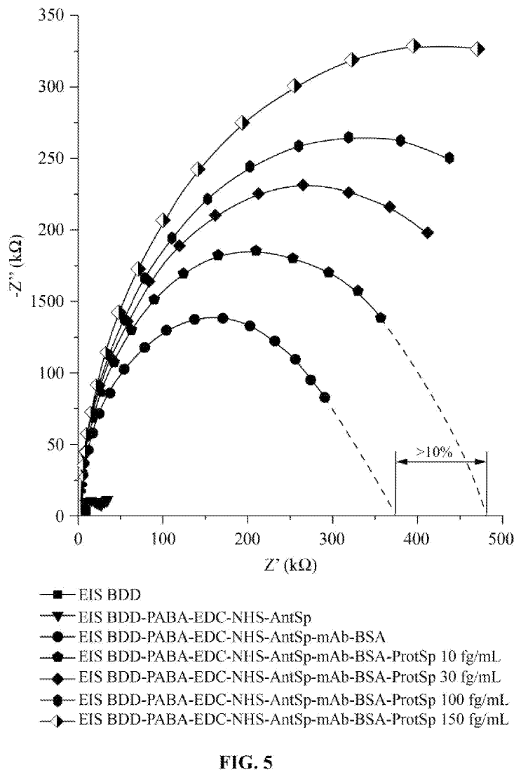

In a particular example and regarding FIG. 5, the electrochemical measurement data of different samples which are generated by the computing unit (6) can be observed in a Nyquist plot. The above electrochemical measurement data, which is represented in the Nyquist plot, was obtained by applying equations 2 and 3, which were indicated before, to the electrochemical response signal generated by the first electrode (8), where different impedance measurements were made for different scenarios. Specifically, the graph shows the impedance of a sample without target analytes and other samples with target analytes with different concentrations.

In another example of the disclosure, electrochemical measurement data can be obtained by interpolating the discrete points of the Nyquist plot with an equivalent circuit model of the electrochemical cell (e.g., Randles model, simplified Randles model, etc.) to determine Rct and the other parameters directly from the equation representing the equivalent circuit.

On the other hand, in one embodiment of the disclosure, the computing unit (6) can determine the presence of the target analyte solely by using the electrochemical measurement data. The computing unit (6) can assess whether the target analyte is present by calculating the difference between the charge transfer resistance (Rct) of the impedance spectra obtained from the functionalized or biomodified electrode with and without the analyte sample.

In a particular example and consistent with FIG. 5, to determine whether the target analyte is present in the sample being analyzed, a comparison is made between the cross points on the real impedance axis of a sample without target analyte, representing predetermined data, and the cross points on the real impedance axis of a sample to be analyzed, representing electrochemical measurement data, if the cross point on the real impedance axis of the sample to be analyzed is greater than the cross point on the real impedance axis of the sample without target analyte then the sample to be analyzed is considered to be positive. Regarding FIG. 5 and a particular example, when the Rct of the blank measurement (without patient sample) and the Rct of the measurement with patient sample is >10% the sample is considered to be positive, i.e., that the target analyte is present.

### Example 1

A device for electronic and electrochemical measurement of analyte concentrations in biological samples (1) was designed, the features of the device are as follows:

The device for electronic and electrochemical measurement of analyte concentrations in biological samples (1) had the first electrode (8), the second electrode (9), the third electrode (10) and the fourth electrode (11). The first electrode (8) was a biomodified electrode for binding specific analytes of the biological sample, specifically the first electrode (8) is a conductive doped graphene ink on a substrate (13) of silica, a layer of chemically related ligands to bind on one end to the substrate and at the other end a protein via a peptide bond (in one implementation, the diazonium salt mixture of 4-aminobenzoic acid), an antibody that binds to the ligand surface via a peptide bond, and specifically to a protein of the target analyte, in this case a structural protein of SARS COV-2. Moreover, the first electrode (8) had a low molecular weight fluorophore, which reacts to the presence of a SARS COV-2 structural protein.

The computing unit (6) is a Raspberry PI, while an XR2206 integrated circuit was used for the signal generator (4). In turn, the analog-to-digital converter (5) is an MCP3008 integrated circuit.

The device for electronic and electrochemical measurement of analyte concentrations in biological samples (1) had an emitter (14) which was an LED and a detector (15) which was a light intensity sensor, wherein the LED was used to emit radiation at an infrared and visible wavelength. The emitter (14) and detector (15) were coupled in such a way that they were located under the substrate (13).

On the other hand, the electronic signal-conditioning module (3) consisted of an input-conditioning circuit (3A) which had three operational amplifiers, a first operational amplifier which was a compensated non-inverting differential amplifier, a second amplifier which was a voltage comparator and a third operational amplifier which was a voltage follower. Moreover, the electronic signal-conditioning module (3) consisted of an input-selector circuit (7A), said input-selector circuit (7A) being connected to the second electrode (9) and with the fourth electrode (11). In particular, the input-selector circuit (7A) featured a voltage inverter.

Moreover, the electronic signal-conditioning module (3) consisted of an output-conditioning circuit (3B) which had two operational amplifiers, a first operational amplifier which was a gain-controlled integrator and a second operational amplifier which was a compensated non-inverting differential amplifier. Moreover, the electronic signal-conditioning module (3) consisted of an output-selector circuit (7B), said output-selector circuit (7B) being connected to the first electrode (8) and with the detector (15). In particular, the output-selector circuit (7B) featured a voltage inverter.

With the above device, it was possible to combine the electrochemical technique, the field-effect technique, and the electro-optical technique, obtaining more accurate results compared to the results of each individual technique.

1. A device for electronic and electrochemical measurement of analyte concentrations in biological samples (1), comprising:
a first electrode (8) and a second electrode (9);
at least one selector circuit (7) connected to the first electrode (8) and second electrode (9);
an electronic signal-conditioning module (3) connected to at least one selector circuit (7);
an analog-to-digital converter (5) connected to the electronic signal-conditioning module (3); and
a computing unit (6) connected to the analog-to-digital converter (5) and the electronic signal-conditioning module (3), said computing unit (6) controlling at least one selector circuit (7);
wherein the computing unit is configured to transmit electrical signals to the electronic signal-conditioning module (3);
wherein at least one selector circuit (7) is configured to select from the electrodes;
wherein the first electrode (8) is configured to bind specific analytes of the biological sample.

2. The device according to claim 1, further comprising a third electrode (10) connected to at least one selector circuit (7) or to the electronic signal-conditioning module (3).

3. The device according to claim 2, further comprising a fourth electrode (11) connected to at least one selector circuit (7), said fourth electrode (11) being connected to the first electrode (8) and to ground.

4. The device according to claim 2, further comprising a Faraday cage (2) enclosing the first electrode (8), the second electrode (9), and the third electrode (10).

5. The device according to claim 1, wherein the at least one selector circuit (7) is selected from the group consisting of an analog or digital demultiplexer, an analog or digital multiplexer, a transistor (e.g., BJT transistors, MOSFETS transistors, IGBT transistors), a solid-state relay, a logic gate, a thyristor, a Silicon-Controlled Rectifier (SCR), a triac diode, a diac diode, an optocoupler, or combinations thereof.

6. The device according to claim 1, wherein the computing unit (6) has a signal generator (4), said signal generator (4) being configured to transmit some voltage signals to the signal-conditioning module (3).

7. The device according to claim 1, wherein the first electrode (8) and the second electrode (9) are placed on a substrate (13).

8. The device according to claim 2, wherein the first electrode (8), the second electrode (9) and the third electrode (10) are placed on a substrate (13); and wherein the first electrode (8) and the third electrode (10) are printed with a conductive carbon or graphene oxide ink and the second electrode (9) with a conductive Ag/AgCl ink on the substrate (13).

9. The device according to claim 1, wherein the electronic signal-conditioning module (3) comprises: an input-conditioning circuit (3A) connected to an input-selector circuit (7A), said input-selector circuit (7A) being connected to the second electrode (9); and an output-conditioning circuit (3B) connected to an output-selector circuit (7A), said output-selector circuit (7B) being connected to the first electrode (8).

10. The device according to claim 3, wherein the electronic signal-conditioning module (3) comprises: an input-conditioning circuit (3A) connected to an input-selector circuit (7A), said input-selector circuit (7A) being connected to the second electrode (9), the third electrode (10), and the fourth electrode (11); and an output-conditioning circuit (3B) connected to an output-selector circuit (7A), said output-selector circuit (7B) being connected to the first electrode (8).

11. The device according to claim 3, wherein the computing unit (6) is connected to an emitter (14) and a detector (15), said detector (15) being connected to the electronic signal-conditioning module (3);
wherein the emitter (14) is configured to emit radiation at a given wavelength and the detector (15) is configured to receive a response radiation from the interaction of the radiation emitted by the emitter (14) with the first electrode (8) and to generate a voltage signal from the response radiation and deliver it to the electronic signal-conditioning module (3).

12. The device according to claim 11, wherein the electronic signal-conditioning module (3) comprises: an input-conditioning circuit (3A) connected to an input-selector circuit (7A), said input-selector circuit (7A) being connected to the second electrode (9), the third electrode (10) and the fourth electrode (11); and an output-conditioning circuit (3B) connected to an output-selector circuit (7A), said output-selector circuit (7B) being connected to the first electrode (8) and to the detector (15).

13. A method for electronic and electrochemical measurement of analyte concentrations in biological samples, comprising:
a) receiving a first switching signal in at least one selector circuit (7), said selector circuit (7) selecting a first electrode (8) and a second electrode (9), said first switching signal being generated by a computing unit (6) connected to the selector circuit (7);
b) applying first electrical signals to the first electrode (8) and the second electrode (9), said first electrical signals being transmitted by an electronic signal-conditioning module (3), which is connected to the first electrode (8), the second electrode (9), and the computing unit (6).
c) generating an electrical signal of electrochemical response at the first electrode (8) from the first electrical signals in the presence of a given concentration of analytes;
d) receiving a second switching signal in at least one selector circuit (7), said selector circuit (7) selecting the first electrode (8) and disconnecting the second electrode (9), said second switching signal being generated by the computing unit (6) connected to the selector circuit (7);
e) applying a second electrical to the first electrode (8), said second voltage signals being generated by the computing unit (6);
f) generating a field-effect response electrical signal at the first electrode (8) in the presence of a given concentration of analytes;
g) generating final measurement data from the electrical response signals generated in steps c) and f) in the computing unit (6).

14. The method according to claim 13, further comprising a third electrode (10) connected to the selector circuit (7);
wherein, in step a) the selector circuit (7) selects the first electrode (8), the second electrode (9) and the third electrode (10) and in step e) the selector circuit (7) selects the first electrode (8) and the third electrode (10) and disconnects the second electrode (9).

15. The method according to claim 14, further comprising a fourth electrode (11) connected to the selector circuit (7);
wherein, in step a) the selector circuit (7) selects the first electrode (8), the second electrode (9) and the third electrode (10) and in step e) the selector circuit (7) selects the first electrode (8), the third electrode (10) and the fourth electrode (11) and disconnects the second electrode (9).

16. The method according to claim 13, further comprising the following steps:
activating an emitter (14) to emit radiation with a given wavelength onto the surface of the first electrode (8);
detecting intensity changes in the expected frequency of fluorescence by means of a detector (15); and
generating an electrical signal of electro-optical response from the detected intensity changes;
wherein, in step i) the electrical signal of electro-optical response is considered to generate the final measurement data.

17. The method according to claim 13, wherein the detector (15) is connected to the at least one selector circuit (7),
wherein, when the control circuit (7) receives a third switching signal, said selector circuit (7) selects the detector (15) and switches off the first electrode (8) and the second electrode (9).

18. The method according to claim 13, wherein the detector (15) is connected to the at least one selector circuit (7),
wherein, when the control circuit (7) receives a third switching signal, said selector circuit (7) selects the detector (15) and switches off the first electrode (8), the second electrode (9), the third electrode (10), and the fourth electrode (11).
